(19) United States
(12) Patent Application Publication
(10) Pub. No.: US 2025/0170246 A1
(43) Pub. Date: May 29, 2025

(54) CDP-CHOLINE A HOST-DIRECTED THERAPEUTIC FOR DISEASE CAUSED BY SARS COV-2 INFECTION

(71) Applicant: Ohio State Innovation Foundation, Columbus, OH (US)

(72) Inventors: Ian Christopher Davis, Hilliard, OH (US); Jean E. Schelhorn, Granville, OH (US)

(21) Appl. No.: 18/840,258

(22) PCT Filed: Mar. 7, 2023
(86) PCT No.: PCT/US2023/063839

§ 371 Date: Aug. 21, 2024

Related U.S. Application Data
(60) Provisional application No. 63/269,549, filed on Mar. 18, 2022.

(51) Int. Cl. A61K 47/54; A61K 31/573; A61P 31/14

(52) U.S. Cl. CPC A61K 47/549; A61K 31/573; A61P 31/14

(57) ABSTRACT

Compositions and methods are disclosed for treating coronavirus infections, such as SARS CoV-2 coronavirus infections. For example, a composition is disclosed that contains one, two, or more cytidine diphosphate (CDP)-conjugated phospholipid precursors selected from the group consisting of CDP-choline (CDP-CHO), CDP-ethanolamine (CDP-ETH), and CDPdiacylglycerol (CDP-DAG) in combination with one or more agents for treating COVID-19, such as corticosteroids, antibodies, or antivirals, in a pharmaceutically acceptable carrier. Also disclosed is a method of treating coronavirus (e.g. SARS CoV-2) infection in a subject that involves administering to the subject one, two, or more cytidine diphosphate (CDP)-conjugated phospholipid precursors selected from the group consisting of CDP-choline (CDP-CHO), CDPethanolamine (CDP-ETH), and CDP-diacylglycerol (CDP-DAG) in combination with agents for treating COVID-19, such as corticosteroids, antibodies, or antivirals.

### CROSS-REFERENCE TO RELATED APPLICATIONS

This application claims benefit of U.S. Provisional Application No. 63/269,549, filed Mar. 18, 2022, which is hereby incorporated herein by reference in its entirety.

### BACKGROUND OF THE INVENTION

Since its emergence in Wuhan, China in December 2019 (Zhou P, et al. Nature. 2020 579(7798):270-3), the SARS CoV-2 coronavirus has rapidly spread across the globe. The virus is highly contagious and to date has infected >30 million Americans, resulting in >550,000 deaths and counting. Although effective vaccines are now available (Thompson M G, et al. MMWR Morb Mortal Wkly Rep. 2021 70(13):495-500), vaccine uptake is sub-optimal (Robinson E, et al. Vaccine. 2021. Epub 2021 Mar. 17), and concerns remain about their ability to protect against novel, potentially more transmissible CoV-2 mutants (Supasa P, et al. Cell. 2021. Epub 2021 Mar. 22; Davies N G, et al. Science. 2021. Epub 2021 Mar. 5). Hence, socioeconomically disruptive infection control measures such as basic hygiene procedures and social distancing remain necessary (Nicola M, et al. Int J Surg. 2020 77:206-16; Nicola M, et al. Int J Surg. 2020 78:185-93).

Severely ill SARS CoV-2-infected patients develop COVID-19 (Li X, et al. 2020 24(1):198; Gattinoni L, et al. Am J Respir Crit Care Med. 2020 201(10):1299-300; Rodriguez-Morales A J, et al. Trav Med Infect Dis. 2020:101623). The mortality rate in confirmed CoV-2 cases is higher than for influenza A virus (IAV) and exceeds 30% for those admitted to the ICU (Auld S, et al. medRxiv. 2020:2020.04.23.20076737). Although systemic dexamethasone and tocilizumab may be beneficial in critically ill patients with COVID-19 (Horby P, et al. N Engl J Med. 2021 384(8):693-704; Gordon A C, et al. N Engl J Med. 2021. Epub 2021 Feb. 26; Jamaati H, et al. Eur J Pharmacol. 2021 897:173947; Rosas 10, et al. N Engl J Med. 2021. Epub 2021 Feb. 26), other treatment options are limited and most patients rely on supportive ICU care (Matthay M A, et al. J Clin Invest. 2012 122(8):2731-40; Hough C L, et al. Curr Opin Crit Care. 2012 18(1):8-15) which is resource-intensive, often ineffective, and not without its own risks (Dhanireddy S, et al. Lab Invest. 2006 86(8):790-9; Bates J H T, et al. Ann Transl Med. 2018 6(19):378). Approximately 20% of all hospitalized COVID-19 patients (Rosenthal N, et al. JAMA Netw Open. 2020 3(12):e2029058) and over 40% of those admitted to the ICU who require mechanical ventilator support die (Di Fusco M, et al. J Med Econ. 2021; 24(1):308-17). Moreover, around 15% of survivors (Sudre C H, et al. Nat Med. 2021. Epub 2021 Mar. 12) develop “long COVID” (Nalbandian A, et al. Nat Med. 2021 27:601-15). Hence, additional medical countermeasures to flatten the curve and improve outcomes for patients with COVID-19 are urgently needed.

### SUMMARY OF THE INVENTION

The liponucleotides (LPNs) CDP-choline, CDP-ethanolamine, and CDP-diacylglycerol are essential precursors (cytidine diphosphate (CDP)-conjugated liponucleotide precursors) for de novo production of all phospholipids in all species and LPN synthesis is rate limiting for this process. LPN synthesis by alveolar type II (ATII) respiratory epithelial cells is rapidly and completely inhibited by influenza A virus infection in mice. Both post-infection LPN prophylaxis and/or treatment of late-stage influenza-induced acute respiratory distress syndrome (ARDS) with a single dose of LPNs can attenuate or even prevent development of hypoxemia, improve lung function, and reduce pulmonary inflammation. See WO2018/005527, which is incorporated by reference in its entirety for the description of CDP-conjugated phospholipid precursors and their use in treating ARDS.

In addition to its extrapulmonary complications, infection with SARS CoV-2 coronavirus can result in lung dysfunction and injury. To determine if CDP-conjugated phospholipid could be beneficial in COVID-19 patients, a mouse model (K18-hACE2-transgenic mice) was used to show that infection with SARS CoV-2 virus resulted in hypoxemia in male but not female mice that was reversed by daily post-infection treatment with CDP-choline. CDP-choline administration also attenuated SARS CoV-2-induced bradycardia and reduced viral replication by 0.5 logs. CDP-choline treatment also reduced whole lung kc/cxcl-1, il-6, and ccl-3/mip-1a gene expression by >5-fold in CoV-2-infected male K18-hACE2-Tg mice. This indicates a significant beneficial effect on cardiopulmonary function together with significant antiviral and anti-inflammatory effects. CDP-choline has no such antiviral effect against influenza.

CDP-choline treatment also attenuated production of the pro-inflammatory mediator IL-8/CXCL-1 by SARS CoV-2-infected human precision cut lung slices. This suggests that the anti-inflammatory effects of CDP-choline are not restricted to mice and provide early evidence that CDP-choline could be of value both as an antiviral agent and as a therapeutic for SARS CoV-2-induced cardiopulmonary dysfunction and pulmonary and systemic inflammation. The antiviral activity of CDP-choline against SARS CoV-2 is an unprecedented finding.

Compositions and methods are therefore disclosed for treating coronavirus infections, such as SARS CoV-2 coronavirus infections. For example, a composition is disclosed that contains one, two, or more cytidine diphosphate (CDP)-conjugated phospholipid precursors selected from the group consisting of CDP-choline (CDP-CHO), CDP-ethanolamine (CDP-ETH), and CDP-diacylglycerol (CDP-DAG) in combination with one or more corticosteroids in a pharmaceutically acceptable carrier. Also disclosed is a method of treating coronavirus (e.g., SARS CoV-2) infection in a subject that involves administering to the subject one, two, or more cytidine diphosphate (CDP)-conjugated phospholipid precursors selected from the group consisting of CDP-choline (CDP-CHO), CDP-ethanolamine (CDP-ETH), and CDP-diacylglycerol (CDP-DAG) in combination with one or more agents for treating COVID-19, such as corticosteroids, antibodies, or antivirals.

Examples of corticosteroids include betamethasone, budesonide, cortisone, dexamethasone, hydrocortisone, methylprednisolone, prednisolone, prednisone, and triamcinolone.

Diacylglycerol (DAG) is a glyceride consisting of two fatty acid chains covalently bonded to a glycerol molecule through ester linkages. Two possible forms exist, 1,2-diacylglycerols and 1,3-diacylglycerols. In some embodiments, the CDP-DAG contains short-chain fatty acids (with aliphatic tails containing fewer than 6 carbons), medium-chain fatty acids (with aliphatic tails containing 6-12 carbons), long-chain fatty acids (with aliphatic tails containing 13-21 carbons), or very long-chain fatty acids (with aliphatic tails containing more than 22 carbons). Fatty acids may be of natural origin or generated by chemical synthesis, according to any methods known to those skilled in the art. In some embodiments, the two fatty acid chains are in the 1,2 positions. In some embodiments, the two fatty acid chains are in the 1,3 positions. In some embodiments, both fatty acid chains are of the same length (contain the same number of carbons). In some embodiments, the two fatty acid chains are of different lengths. In some embodiments, one or both fatty acid chains of the DAG component of CDP-DAG are mono-unsaturated (containing one double bond in cis and/or trans configuration). In some embodiments, one or both fatty acid chains of the DAG component of CDP-DAG are poly-unsaturated (containing more than one double bond in cis and/or trans configuration). In some embodiments, one or both fatty acid chains of the DAG component of CDP-DAG are saturated (containing no double bonds). In some embodiments, one or both fatty acid chains are chemically modified. Chemical modifications include, but are not limited to, methylation, esterification, amidation, nitration, nitrosylation, oxidation, sulfation, acetylation, alcoholysis, acidolysis, biotinylation, conjugation to fluorophores, and other modifications known to those skilled in the art.

In some embodiments, the CDP component of CDP-CHO is chemically modified. Chemical modifications include, but are not limited to, methylation, esterification, amidation, nitration, nitrosylation, oxidation, sulfation, acetylation, alcoholysis, acidolysis, biotinylation, conjugation to fluorophores, and other modifications known to those skilled in the art.

In some embodiments, the CDP component of CDP-ETH is chemically modified. Chemical modifications include, but are not limited to, methylation, esterification, amidation, nitration, nitrosylation, oxidation, sulfation, acetylation, alcoholysis, acidolysis, biotinylation, conjugation to fluorophores, and other modifications known to those skilled in the art.

In some embodiments, the CDP component of CDP-DAG is chemically modified. Chemical modifications include, but are not limited to, methylation, esterification, amidation, nitration, nitrosylation, oxidation, sulfation, acetylation, alcoholysis, acidolysis, biotinylation, conjugation to fluorophores, and other modifications known to those skilled in the art.

In some embodiments, the choline component of CDP-CHO is chemically modified. Chemical modifications include, but are not limited to, methylation, esterification, amidation, nitration, nitrosylation, oxidation, sulfation, acetylation, alcoholysis, acidolysis, biotinylation, conjugation to fluorophores, and other modifications known to those skilled in the art.

In some embodiments, the ethanolamine component of CDP-ETH is chemically modified. Chemical modifications include, but are not limited to, methylation, esterification, amidation, nitration, nitrosylation, oxidation, sulfation, acetylation, alcoholysis, acidolysis, biotinylation, conjugation to fluorophores, and other modifications known to those skilled in the art.

In some embodiments, the glycerol component of CDP-DAG is chemically modified. Chemical modifications include, but are not limited to, methylation, esterification, amidation, nitration, nitrosylation, oxidation, sulfation, acetylation, alcoholysis, acidolysis, biotinylation, conjugation to fluorophores, and other modifications known to those skilled in the art.

In some embodiments, a mixture of two or more CDP-CHO-derived Plipid precursors with or without different chemical modifications of CDP and/or choline can be incorporated.

In some embodiments, a mixture of two or more CDP-ETH-derived Plipid precursors with or without different chemical modifications of CDP and/or ethanolamine chains can be incorporated.

In some embodiments, a mixture of two or more CDP-DAG-derived Plipid precursors with or without different acylations or chemical modifications of CDP and/or fatty acid chains can be incorporated.

In some embodiments, the CDP-conjugated Plipid precursors are collectively present at a unit dose of at least 0.1 ng/kg, including 0.1, 0.2, 0.3, 0.4, 0.5, 0.6, 0.7, 0.8, 0.9, 1.0 ng/kg.

In some embodiments, the CDP-CHO and/or CDP-ETH and/or CDP-DAG are present in equal concentrations or ratios. In some embodiments, at least two of the CDP-conjugated Plipid precursors are present in equal concentrations or ratios, which can be higher or lower than the third CDP-conjugated Plipid precursor, which may be absent. In some cases, one of the CDP-conjugated Plipid precursors is present at a concentration or ratio that is at least 2, 3, 4, 5, 6, 7, 8, 9, or 10 fold higher than one or both of the other CDP-conjugated Plipid precursors.

The disclosed compositions can further contain other active and inactive ingredients. For example, in some embodiments, the composition can contain additional lipid moieties, nucleotides, organic acids, amino acids, or sugars. The composition can also contain a stabilizer.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 illustrates how SARS CoV-2 infection may disrupt de novo phosphatidylcholine synthesis in human ATII cells, resulting in ATII cell dysfunction.

FIG. 2 shows de novo phosphatidylcholine synthesis by the Kennedy Pathway.

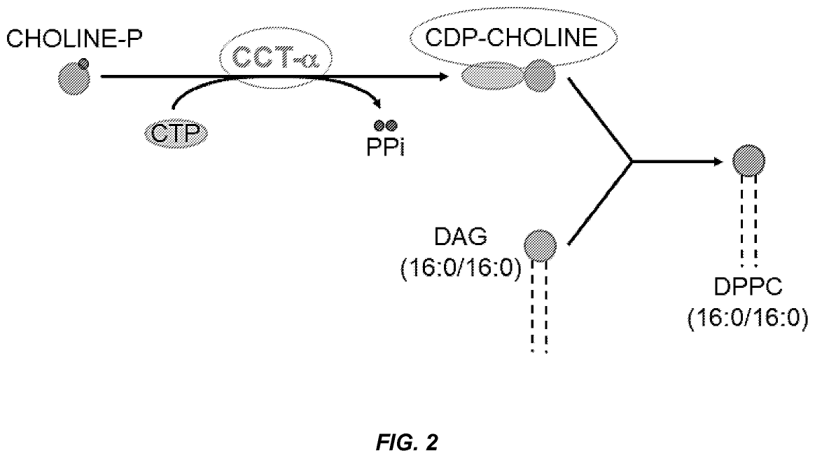

FIGS. 3A and 3B show effect of intranasal mock-infection and infection of 8-12 week-old male K18-hACE2-Tg mice with 105 TCID50 SARS CoV-2 (WA1 strain) and daily post-infection treatment with 50 μl saline i.p. or CDP-choline (5 mg/kg, i.p. in 50 μl saline) on (A) Carotid arterial % 02 saturation (SaO2) and (B) Heart rate (in beats per minute) at 4 days post-inoculation. *: P<0.05, **: P<0.005, vs. Mock.

FIG. 4 shows effect of daily post-infection treatment with 50 μl saline i.p. or CDP-choline (5 mg/kg, i.p. in 50 μl saline) on SARS CoV-2 (WA1 strain) replication in lungs of 8-12 week-old male K18-hACE2-Tg mice at 4 days post-inoculation. **: P<0.005, vs. Saline.

FIG. 5 shows effect of daily post-infection treatment with CDP-choline (5 mg/kg, i.p. in 50 μl saline) on expression of keratinocyte cytokine (KC/CXCL1) and Interleukin-6 (IL-6) in lungs of 8-12 week-old male K18-hACE2-Tg mice infected intranasally with 105 TCID50 SARS CoV-2 (WA1 strain) at 4 days post-inoculation, shown as fold-change relative to infected mice treated daily with 50 μl saline i.p. only. Dotted line indicates 2-fold change, considered cutoff for biological significance.

FIG. 6 shows effect of daily post-infection addition of 50 μl saline or 50 μl saline containing 100 μM CDP-choline on levels of Interleukin-8 (IL-8) protein in culture supernatants from human precision-cut lung slices that were mock-infected or infected with 4×105 TCID50 SARS CoV-2 (WA1 strain). †: P<0.05, vs. Saline.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal or bird. Thus, the subject can be a human or veterinary patient. The term “patient” refers to a subject under the treatment of a clinician, e.g., physician or veterinarian, as well as other allied health professionals, including (but not limited to) nurses, physician's assistants, and pharmacists.

The term “therapeutically effective” refers to the amount of the composition used is of sufficient quantity to ameliorate one or more causes, symptoms, and/or clinical signs of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms and/or clinical signs rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Coronaviruses are a group of related RNA viruses that cause diseases in mammals and birds. In humans and birds, they cause respiratory tract infections that can range from mild to lethal. Coronaviruses constitute the subfamily Orthocoronavirinae, in the family Coronaviridae, order Nidovirales, and realm Riboviria. Coronaviruses are divided into the four genera: Alphacoronavirus, Betacoronavirus, Gammacoronavirus and Deltacoronavirus. Alphacoronaviruses and betacoronaviruses infect mammals, while gammacoronaviruses and deltacoronaviruses primarily infect birds. Therefore, in some embodiments, the coronavirus is an alphacoronavirus or betacoronavirus.

Alphacoronaviruses (Alpha-CoV) that infect humans include Human coronavirus 229E (HCoV-229E) and Human coronavirus NL63 (HCoV-NL63).

Betacoronaviruses (BetaCoVs) comprise four varying viral lineages: A, B, C, D. The betacoronaviruses of the greatest clinical importance concerning humans are Human coronaviruses OC43 and HKU1 of lineage A, severe acute respiratory syndrome coronavirus (SARS-CoV) and SARS-CoV-2 (which causes the disease coronavirus disease 2019 [COVID-19]) of lineage B, and Middel East respiratory syndrome coronavirus (MERS-CoV) of lineage C. In some embodiments, the betacoronavirus is a lineage A, B, C, or D betacoronavirus. In some embodiments, the disclosed compositions and methods can be used to treat any betacoronavirus. In some embodiments, the disclosed compositions and methods can be used to treat a betacoronavirus of genus A, B, C, or D. Therefore, in some embodiments, the disclosed compositions and methods can be used to treat a coronavirus, such as SARS-CoV-2.

Cytidine diphosphate-choline (CDP-choline) is a naturally occurring compound that is synthesized from cytidine-5′-triphosphate and phosphocholine with accompanying production of inorganic pyrophosphate in a reversible reaction catalyzed by the enzyme CTP:phosphocholine cytidylyltransferase-α (pcyt1a). CDP-ethanolamine is synthesized from cytidine-5′-triphosphate and phosphoethanolamine with accompanying production of inorganic pyrophosphate in a reversible reaction catalyzed by the enzyme CTP-phosphoethanolamine cytidyltransferase (pcyt2).

The molecular structure of CDP-choline is provided below.

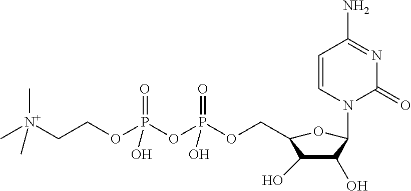

The molecular structure of CDP-ethanolamine is provided below.

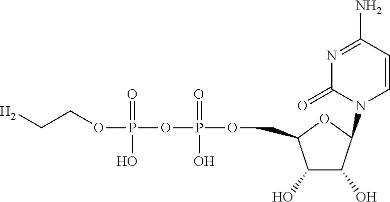

Molecular structures of CDP-DAG are provided below.

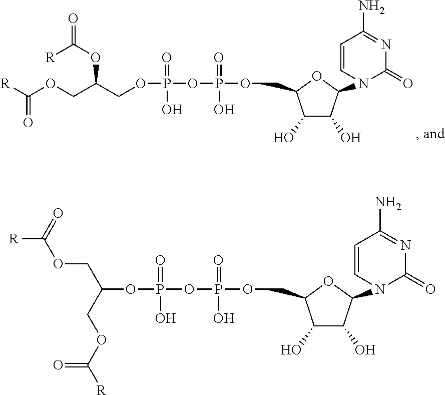

In these structures, R denotes points of attachment of various length acyl chains to the glycerol moiety of CDP-DAG.

The compositions disclosed can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans or animals, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients, such as antimicrobial agents, anti-inflammatory agents, anesthetics, vaccine antigens, adjuvants, PAMPs, and DAMPs,

Preparations for enteral and/or parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Enteral and parenteral vehicles include sodium chloride solution, Ringer's dextrose, dextrose and sodium chloride, lactated Ringer's, glucose, or fixed oils. Intravenous vehicles include fluid and nutrient replenishers, electrolyte replenishers (such as those based on Ringer's dextrose), and the like. Mucosal vehicles include sterile aqueous or non-aqueous solutions, suspensions, and emulsions. Examples include sodium chloride solution, Ringer's dextrose, dextrose and sodium chloride, lactated Ringer's, glucose, fixed oils, propylene glycol, and the like. Preservatives and other additives may also be present such as, for example, antimicrobials, anti-oxidants, chelating agents, and inert gases.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The herein disclosed compositions, including pharmaceutical composition, may be administered in a number of ways depending on whether the desired treatment is prophylactic, for prevention of development of COVID-19 in SARS CoV-2-exposed, and/or SARS CoV-2-infected, and/or other at-risk persons, or for acute treatment of persons with COVID-19. For example, the disclosed compositions can be administered orally in powder or tablet form for prophylaxis and prevention of COVID-19 or given intravenously, intraperitoneally, intramuscularly, subcutaneously, intracavity, or transdermally for treatment of COVID-19. Pharmaceutical grade compositions may be administered orally as a compounded tablet including active ingredients at appropriate doses, excipients, and coatings for easing swallowing, and/or controlling release rate of active ingredients, and for shelf life extension. Pharmaceutical grade compositions may be administered orally as a liquid suspension or emulsion. Pharmaceutical grade compositions may be administered parenterally (e.g., intravenously with appropriate carriers, and stabilizers), by intramuscular injection, by intraperitoneal injection, transdermally, extracorporeally, ophthalmically, vaginally, rectally, intranasally, topically or the like, including topical intranasal administration or administration by inhalant.

In one embodiment, the disclosed compositions are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of the disclosed compositions administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Alveolar type II (ATII) cells are essential to normal alveolar gas exchange and lung function (Mason R J. Respirology. 2006 11 Suppl:S12-S5). Human ATII cells express the SARS CoV-2 receptor (angiotensin converting enzyme 2 [ACE2]) (Zou X, et al. Front Med. 2020. Epub 2020 Mar. 15) and TMPRSS2 protease (Hoffmann M, et al. Cell. 2020 181(2):271-80.e8). SARS CoV-2 antigens have been detected in ATII cells in COVID-19 autopsy samples (Puelles V G, et al. New Engl J Med. 2020. Epub 2020 May 14; Adachi T, et al. Emerg Infect Dis. 2020 26(9)) and in experimental models (Rockx B, et al. bioRxiv. 2020:2020.03.17.995639; Chu H, et al. Clin Infect Dis. 2020. Epub 2020 Apr. 10; Yu P, et al. Animal Model Exp Med. 2020 3(1):93-7; Hekman R M, et al. Molecular Cell. 2020 80(6):1104-22; Pei R, et al. Protein Cell. 2020:1-17; Youk J, et al. Cell Stem Cell. 2020 27(6):905-19). Hence, they are likely to be central players in COVID-19 pathogenesis (Mason R J. Eur Respir J. 2020 55(4):2000607).

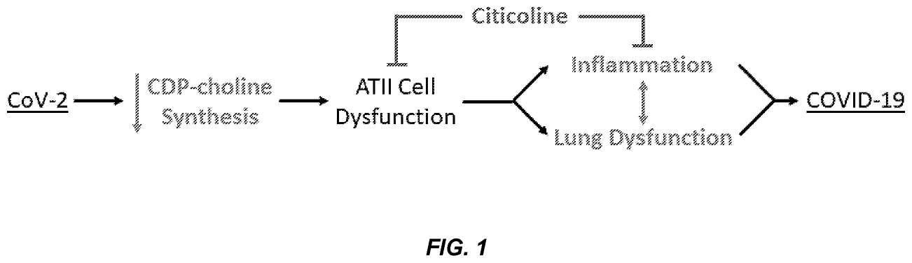

It is proposed herein that CoV-2 infection disrupts de novo phosphatidylcholine synthesis in human ATII cells, resulting in ATII cell dysfunction (See FIG. 1). This promotes a vicious cycle of lung dysfunction and inflammation and leads to progression to COVID-19. Bypassing the block in phosphatidylcholine synthesis by administration of exogenous CDP-choline (as citicoline) to patients can directly (via effects on ATII cells) or indirectly (through effects on innate immune cells) break the cycle and thereby safely improve COVID-19 patient outcomes.

Citicoline has efficacy in preclinical models of both IAV-induced ARDS (Rosas L E, et al. Am J Respir Cell Mol Biol. 2021. Epub 2021 Feb. 20) and COVID-19. Citicoline is a well-understood, commercially available small molecule with profound anti-inflammatory effects (Grieb P. CNS Drugs. 2014 28(3):185-93; EFSA Panel on Dietetic Products NaAN. EFSA J. 2013 11(10)) that has been shown to be safe in multiple human clinical trials. However, it has not been used in critically ill patients with COVID-19, who may be at higher risk of adverse effects.

Abbreviations: “ARDS”: Acute respiratory distress syndrome; “ATII cell”: Alveolar type II cell; COVID-19: coronavirus disease 2019; “dpi”: Days post-inoculation; “SAE”: Serious adverse event; SARS CoV-2: severe acute respiratory syndrome coronavirus-2; “SCARLET”: Supplemental Citicoline Administration to Reduce Lung injury Efficacy Trial; “S:F”: SₚO₂:FᵢO₂ ratio.

To date, there have been >78 million SARS CoV-2 coronavirus infections in the US, resulting in >900,000 deaths and counting. Effective vaccines are now available (Thompson M G, et al. MMWR Morb Mortal Wkly Rep. 2021 70(13):495-500), but infection control measures such as enhanced basic hygiene procedures, use of PPE, and social distancing may need to remain in effect for some time, resulting in considerable societal disruption (Nicola M, et al. Int J Surg. 2020 77:206-16).

The mortality rate in confirmed SARS CoV-2 cases is higher than for influenza A virus (IAV) and may be ≥2% (Basu A. Health Affairs. 2020:10.1377/hlthaff.2020.00455). Death generally results from a clinical syndrome termed coronavirus disease of 2019 (COVID-19). This is a somewhat atypical form of the acute respiratory distress syndrome (ARDS), often accompanied by coagulopathy, acute cardiac injury, renal injury, and other systemic manifestations (Li X, et al. 2020 24(1):198; Gattinoni L, et al. Am J Respir Crit Care Med. 2020 201(10):1299-300; Tsolaki V, et al Am J Respir Crit Care Med. 2020; Teuwen L A, et al. Nat Rev Immunol. 2020:1-3). Approximately 20% of all hospitalized patients (Rosenthal N, et al. JAMA Netw Open. 2020 3(12):e2029058) and up to 40% of those admitted to the ICU (Di Fusco M, et al. J Med Econ. 2021; 24(1):308-17) die. Moreover, around 15% of survivors (Sudre C H, et al. Nat Med. 2021. Epub 2021 Mar. 12) develop “long COVID” (Nalbandian A, et al. Nat Med. 2021 27:601-15), a disabling condition which is more likely after severe acute illness (lqbal A, et al. Cureus. 2021 13(2):e13080).

Treatment options for critically ill patients with COVID-19 are limited and most patients rely on supportive ICU care, particularly high concentrations of supplemental O₂ and mechanical ventilation (Matthay M A, et al. J Clin Invest. 2012 122(8):2731-40; Hough C L, et al. Curr Opin Crit Care. 2012 18(1):8-15). Supportive care is resource-intensive and often ineffective, and interventions such as O₂ therapy and mechanical ventilation can themselves be injurious to the lung (Dhanireddy S, et al. Lab Invest. 2006 86(8):790-9; Bates J H T, et al. Ann Transl Med. 2018 6(19):378). Systemic dexamethasone and tocilizumab may (Horby P, et al. N Engl J Med. 2021 384(8):693-704; Gordon A C, et al. N Engl J Med. 2021. Epub 2021 Feb. 26) or may not be beneficial (Jamaati H, et al. Eur J Pharmacol. 2021 897:173947; Rosas 10, et al. N Engl J Med. 2021. Epub 2021 Feb. 26; Chaudhry Z, et al. J Infect. 2021. Epub 2021 Mar. 16) in critically ill patients, while hydroxychloroquine (Singh B, et al. Cochrane Database Syst Rev. 2021 2:Cd013587), convalescent plasma (Janiaud P, et al. JAMA. 2021) and the antiviral drug remdesivir (Spinner C D, et al. JAMA. 2020 324(11):1048-57) are all ineffective. Additional medical countermeasures to flatten the curve and manage patients with COVID-19 are therefore urgently needed.

Approximately 50% of the epithelial cells lining the alveoli in the distal lung are small cuboidal ATII cells (Beers M F, et al. Am J Respir Cell Mol Biol. 2017 57(1):18-27). ATII cells regulate the depth of the alveolar lining fluid by alveolar fluid clearance (Davis I C, et al. Adv Exp Med Biol. 2007 618:127-40). They also synthesize, secrete, and recycle pulmonary surfactant proteins and lipids (including phospholipids), which help to maintain low alveolar surface tension (Whitsett J A, et al. Ann Rev Med. 2010 61(1):105-19), reducing dynamic alveolar collapse and preventing gas exchange impairment during ventilation. Surfactant phospholipids also have anti-inflammatory properties, and surfactant proteins play an important role in host defense against pathogens (Han S, et al. Annals Am Thorac Soc. 2015 12(5):765-74).

It is proposed herein that SARS CoV-2 infection disrupts de novo phosphatidylcholine synthesis in human ATII cells, resulting in ATII cell dysfunction. This promotes a vicious cycle of lung dysfunction and inflammation and leads to progression to COVID-19. Bypassing the block in phosphatidylcholine synthesis by administration of exogenous citicoline will directly (via effects on ATII cells and surfactant function) or indirectly (through effects on innate immune cells) break the cycle of lung injury and gas exchange impairment and thereby safely improve critically ill COVID-19 patient outcomes.

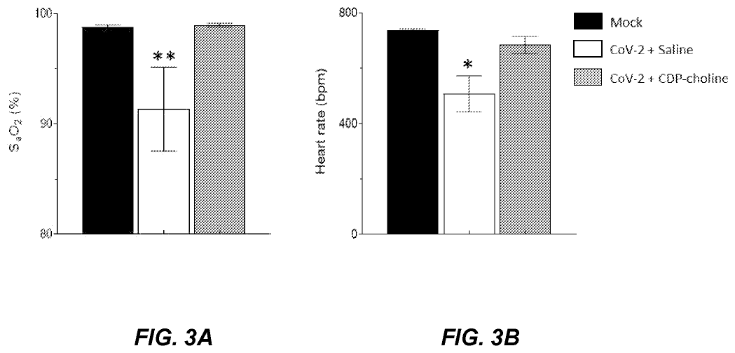

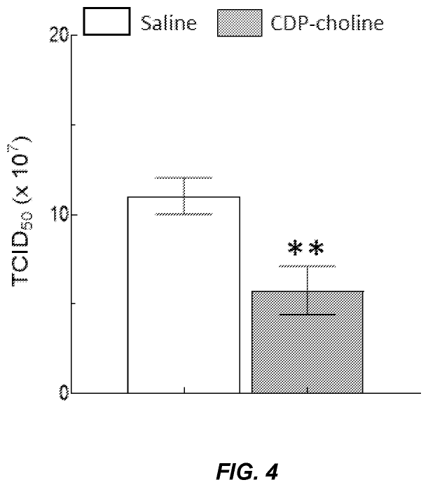

Daily post-infection treatment of SARS CoV-2-infected male K18-hACE2-Tg mice with i.p. CDP-choline prevents development of hypoxemia and bradycardia and significantly reduces viral replication (FIG. 3). CDP-choline also reduced production of pro-inflammatory mediators by SARS CoV-2-infected human precision cut lung slices (FIG. 4). Hence, CDP-choline/citicoline is a COVID-19 therapeutic with both antiviral and host-directed beneficial effects.

In addition to its dramatic impact on oxygenation and pulmonary inflammation, citicoline has many characteristics that would be advantageous to any candidate COVID-19 therapeutic:

Synthesis of CDP-choline is both essential and rate-limiting for de novo phosphatidylcholine synthesis: this pathway is highly conserved across all mammals (Agassandian M, et al. Biochim Biophys Acta. 2013 1831(3):612-25), making it highly likely that beneficial therapeutic effects of citicoline in animal models will be recapitulated in human patients.

CDP-choline promotes phospholipid incorporation into lung tissue (Cetinkaya M, et al. Pediatr Res. 2013 74(1):26-33). CDP-choline also activates CCT-α by promoting its translocation to membranes (Giménez R, et al. Neurosci Lett. 1999 273(3):163-6). CDP-choline that is not incorporated into phospholipids is rapidly metabolized into cytidine and choline, and ultimately excreted in expired CO₂ and via urine (Secades J J. Rev Neurol. 2016 63(S03):S1-S73). Hence, it is unlikely to alter the function of cells in which phospholipid synthesis is not impaired (Dinsdale J R, et al. Arzneimittelforschung. 1983 33(7a):1066-70; Gareri P, et al. Clin Interv Aging. 2015 10:1421-9).

CDP-choline/citicoline is a simple, inexpensive small molecule. Methods for citicoline synthesis are well defined, straightforward, and inexpensive (Ghezal S, et al. Tetrahedron Lett. 2014 55(38):5306-10; Ren Y, et al. Biotechnol Bioeng. 2020 117(5):1426-35). Hence, new stocks can be rapidly generated as existing stockpiles become depleted due to expiration or usage.

Citicoline has high aqueous solubility and excellent bioavailability when administered parenterally (D'Orlando K J, et al. Neurol Res. 1995 17(4):281-4) so does not need to be instilled directly into the lungs. Hence, issues commonly associated with intra-pulmonary drug administration to the injured lung, such as heterogeneity of distribution (Ueda T, et al. J Appl Physiol. 1994 76(1):45-55) and induction of transient hypoxemia (Dushianthan A, et al. Crit Care. 2012 16(6):238) can be avoided. Moreover, i.v. administration will result in distribution to other affected organs in COVID-19 patients, potentially with beneficial anti-inflammatory effects.

Citicoline is profoundly anti-inflammatory (Rosas L E, et al. Am J Respir Cell Mol Biol. 2021. Epub 2021 Feb. 20; Cetinkaya M, et al. Pediatr Res. 2013 74(1):26-33; Yilmaz Z, et al. Shock. 2006 25(1):73-9; Feng X, et al. Int Immunopharmacol. 2020 83:106448; Cetinkaya M, et al. J Surg Res. 2013 183(1):119-28). The apparent beneficial effects of dexamethasone and tocilizumab in COVID-19 (Horby P, et al. N Engl J Med. 2021 384(8):693-704; Gordon A C, et al. N Engl J Med. 2021. Epub 2021 Feb. 26) suggest that other anti-inflammatory drugs (such as citicoline) may likewise be useful. Given its minimal side-effects, citicoline may be superior to corticosteroids, which are harmful in COVID-19 patients not requiring O₂ (Pasin L, et al. J Cardiothorac Vasc Anesth. 2021 35(2):578-84), increase risk for invasive fungal infections (White P L, et al. Clin Infect Dis. 2020. Epub 2020 Aug. 30; Veisi A, et al. Eur J Ophthalmol. 2021:11206721211009450; Sharma S, et al. J Laryngol Otol. 2021:1-6), and are contra-indicated for other severe respiratory coronaviral infections such as SARS-CoV and MERS (Mattos-Silva P, et al. Respir Physiol Neurobiol. 2020 280:103492).

### Results

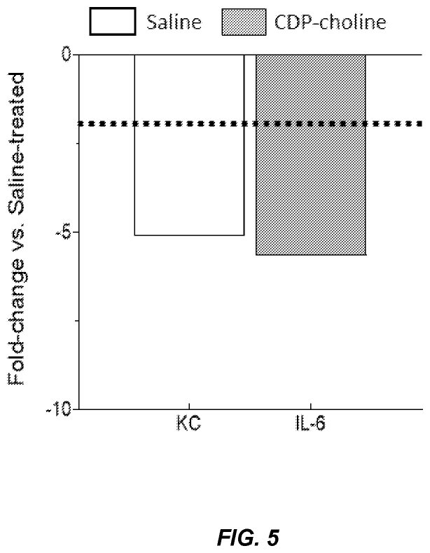

Daily post-infection CDP-choline treatment attenuates SARS CoV-2-induced hypoxemia, bradycardia, and inflammation in vivo. Infection with 10⁵ TCID₅₀ of SARS CoV-2 (WA1 strain) resulted in more rapid weight loss in male than female K18-hACE2-Tg (B6.Cg-Tg(K18-ACE2) 2Prlmn/J) mice (not shown), with deaths from 5 dpi onwards. Infection induced hypoxemia and bradycardia in male but not female K18-hACE2-Tg mice at 4 dpi. Daily i.p. administration of 5 mg/kg CDP-choline from 1-3 dpi completely prevented development of hypoxemia (FIG. 3A) and bradycardia (FIG. 3B) in SARS CoV-2-infected male K18-hACE2-Tg mice at 4 dpi. CDP-choline treatment also significantly decreased SARS CoV-2 replication in the lungs (FIG. 4) and reduced whole lung kc/cxcl-1 and il-6 gene expression by >5-fold in male K18-hACE2-Tg mice (FIG. 5).

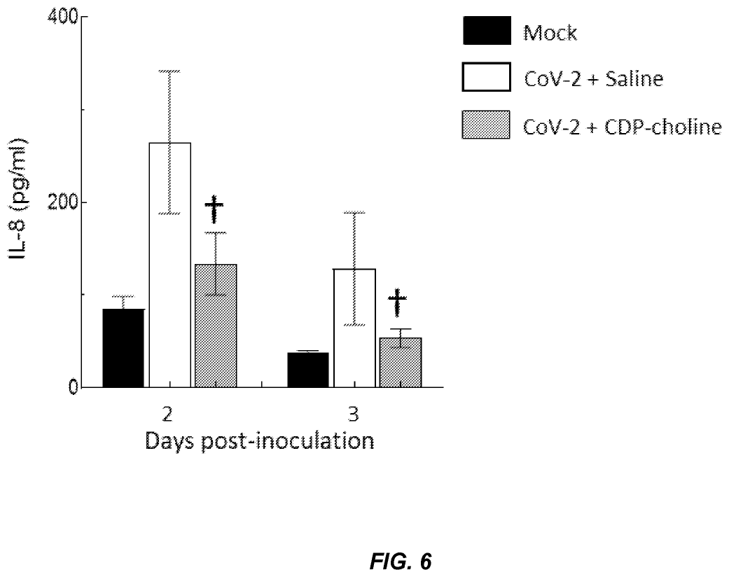

CDP-choline reduces SARS CoV-2-induced inflammation in vitro. Addition of CDP-choline to the culture media significantly reduced production of IL-8 (a putative marker of COVID-19 severity) by precision-cut human lung slices inoculated with 4×10⁵ TCID₅₀ of SARS CoV-2 (WA1 strain) at 2 and 3 dpi (FIG. 6).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a coronavirus infection in a subject, comprising administering to the subject an effective amount of a composition comprising one or more cytidine diphosphate (CDP)-conjugated precursors and administering to the subject an effective amount of an agent for treating COVID-19.

2. The method of claim 1, wherein CDP-conjugated precursors are selected from the group consisting of CDP-choline (CDP-CHO), CDP-ethanolamine (CDP-ETH), CDP-diacylglycerol (CDP-DAG), and combinations thereof.

3. The method of claim 1, wherein the composition comprises two or more CDP-conjugated phospholipid precursors.

4. The method of claim 3, wherein the composition comprises CDP-CHO and CDP-DAG.

5. The method of claim 3, wherein the composition comprises CDP-CHO and CDP-ETH.

6. The method of claim 4, wherein the CDP-CHO and CDP-DAG or CDP-CHO and CDP-ETH are present in equal concentrations.

7. The method of claim 3, wherein the composition comprises CDP-CHO, CDP-ETH, and CDP-DAG.

8. The method of claim 7, wherein the CDP-CHO, CDP-ETH, and CDP-DAG are present in equal concentrations.

9. The method of claim 3, wherein the CDP-conjugated precursors are collectively present at a concentration of at least 0.1 ng per kg of body weight.

10. The method of claim 1, wherein the CDP-conjugated precursors comprise one or more chemical modification selected from the group consisting of methylation, esterification, amidation, nitration, nitrosylation, oxidation, sulfation, acetylation, alcoholysis, acidolysis, biotinylation, and fluorophore conjugation.

11. The method of claim 1, wherein the agent is a corticosteroid is selected from the group consisting of betamethasone, budesonide, cortisone, dexamethasone, hydrocortisone, methylprednisolone, prednisolone, prednisone, and triamcinolone.

12. The method of claim 11, wherein the one or more CDP-conjugated precursors are co-administered with the corticosteroid.

13. The method of claim 11, wherein the one or more CDP-conjugated precursors and corticosteroid are in the same composition.

14. The method of claim 1, wherein the coronavirus is SARS CoV-2 coronavirus.

15. The method of claim 1, wherein the subject is on supplemental oxygen.

16. A composition comprising one or more cytidine diphosphate (CDP)-conjugated precursors and an agent for treating COVID-19.

17. The composition of claim 16, wherein the agent is a corticosteroid.
